SEQUENCE LISTING (4 sequences)

SEQ ID NO: 1 - DNA, 23 bp, Artificial
  synthetic primer for human genome
acagagyragactcyrtctcaac

SEQ ID NO: 2 - DNA, 23 bp, Artificial
  synthetic primer for human genome
acagagyragactcyrtctcaac

SEQ ID NO: 3 - DNA, 20 bp, Artificial
  synthetic primer for human genome
gtgagccaccacgcccagcc

SEQ ID NO: 4 - DNA, 27 bp, Artificial
  synthetic primer for human genome
accacagaattcccaccacgcccagcc

(19) United States
(12) Patent Application Publication
(10) Pub. No.: US 2005/0214834 A1
(43) Pub. Date: Sep. 29, 2005

(54) Methods for eliminating repeat sequences in genome

(76) Inventors: Xin-Yuan Guan, Baguio Villa (HK); Jonathan S.T. Sham, Tam Tower (HK)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/061,719

(22) Filed: Feb. 22, 2005

Related U.S. Application Data
(63) Continuation-in-part of application No. 10/489,759.

(51) Int. Cl. C12Q 1/68

(52) U.S. Cl. 435/6

(57) ABSTRACT

The present invention relates to methods of eliminating repeat sequence elements in a genome, and nucleic acid probes and arrays produced by these methods. The present invention also relates to chromosome-specific DNA probes that are useful in selectively detecting individual chromosomes or chromosome segments, and methods of preparing them. The probes may be used to detect chromosomes or chromosome segments in various methods, including in metaphase cell spreads and interphase nuclei by in situ hybridization. DNA depleted of repeat sequence elements can also be used to construct genome-wide DNA arrays to detect chromosomal arrangements in human cancers.

This application is a continuation-in-part of U.S. Ser. No. 10/489,759 filed Mar. 17, 2004 which claims priority to PCT/CN01/01209 having an international filing date of Jul. 27, 2001, and PCT/CN2002/000580, having an international filing date of Aug. 22, 2002, which are hereby incorporated by reference in their entirety.

### BACKGROUND OF THE INVENTION

Chromosome identification procedures are considered an important aspect of biomedical diagnostic practice because of the many disease syndromes that are associated with or diagnosed by chromosomal aberrations, such as cancers. Being able to observe these chromosomal changes and rearrangements is important for not only diagnostic reasons, but also for therapeutic and prophylactic reasons, e.g., to monitor the efficiency of a therapeutic treatment. Another application of chromosome identification is to monitor the chromosomes of a fetus, especially where the existence of certain fetal risk factors have been identified, such as the age of mother, and exposure to environmental toxins.

### BRIEF DESCRIPTION OF THE DRAWINGS

FIG. 1 shows an illustration of an embodiment of PCR using an Alu 3′-end primer.

FIG. 2. (Upper) BAC clones were amplified with AD2 and PCR products were run in 1% agarose gel and stained with ethidium bromide. At least 2 PCR products were obtained from each BAC clone. The size distribution of PCR products ranged from 400 bp to 3.5 kb. (Lower) The DNA fragments in upper gel were Southern blotted and hybridized with ³²P-labeled BAC RP11-497124.

### DESCRIPTION OF THE INVENTION

The present invention relates to methods of eliminating repeat sequence elements in a genome, and nucleic acid probes and arrays produced by these methods. The present invention also relates to chromosome-specific DNA probes that are useful in selectively detecting individual chromosomes or chromosome segments, and methods of preparing them. The probes may be used to detect chromosomes or chromosome segments in various methods, including in metaphase cell spreads and interphase nuclei by in situ hybridization. DNA depleted of repeat sequence elements can also be used to construct genome-wide DNA arrays to detect chromosomal arrangements in human cancers.

Probes produced by methods of the present invention can be especially useful where they are labeled fluorescently for fluorescent in situ hybridization (“FISH”) techniques. These probes can also be referred to as chromosome painting probes, where the labeled probes are utilized to detectably coat (“paint”) whole chromosomes, or specific parts of them.

In addition to cytogenetics applications, probes produced in accordance with the present invention can also be used as probes for nucleic acid arrays (e.g., DNA arrays), Southern blots, dot blots, and any methods that use nucleic acid probes to detect nucleic acids.

The present invention provides methods of preparing nucleic acid probes, such as chromosome-specific nucleic acid probes, comprising at least one of the following steps in any effective order, e.g., contacting a chromosome, chromosome segment or genomic DNA with an end-Alu primer under conditions effective for said primer to hybridize to a human Alu repetitive element, and amplifying the nucleic acid from the inter-repeat regions of said chromosome, chromosome segment or genomic DNA to produce the chromosome-specific nucleic acid probe, wherein said amplifying is accomplished with a primer consisting of said end-Alu primer. These methods can effectively deplete repetitive sequence during the amplification step from the chromosomal or genomic DNA, decreasing background and non-specific binding, thereby increase the accuracy and reliability of the probes when used, e.g., in FISH, and Southern analysis, or in gene chip arrays. These methods can also be referred to as “inter-Alu PCR” since the regions between (“inter”) Alu elements are amplified.

The chromosome segments can be obtained from the genomes of any suitable organism, including bacteria, protista, molluscs, arthropods, insects (e.g., Drosophila), birds, mammals (e.g., human, primates, monkeys, rodents, mice, rats, dogs, cats, sheep, goat, horses, etc.) Appropriate repetitive elements can be used, e.g., with a spacing about 3-4 kb in the genome of interest.

A “nucleic acid probe” is used to indicate the collection of nucleic acid fragments which are produced by the amplification reaction. For instance, if an Alu primer is used to amplify a chromosome segment in the presence of the appropriate enzyme (e.g., Taq polymerase) and substrates (e.g., nucleotides), nucleic acid fragments of varying lengths will be produced that are substantially complementary (within the accuracy of the polymerase reaction) to regions within the chromosome segment. These fragments are complementary to, and thus will hybridize to, the chromosome segment, and thus, under appropriate conditions, can be used as tool (a “probe”) to detect it.

A “chromosome-specific nucleic probe” is a probe that is selective for a certain chromosome. The selectivity (or specificity) is conferred by the amplification reaction when a particular whole chromosome, or a region of it, is utilized as a template for an amplification reaction in accordance with the present invention. The probe is chromosome-specific because it binds substantially to the chromosome (or the targeted region within it), and thus can used to distinguish it from others. The probes can be used in any application where nucleic acid is to be detected, including, FISH, Southern, gene chip arrays, DNA dot blots, etc. The probes can be attached to a substrate, e.g., in an array, or they can be used to detect sequences which are present on a substrate.

The phrase “chromosome segment” refers to a part of the chromosome that can be used as a template for the amplification reaction. A segment can be any region that is less than the whole chromosome, and includes chromosome bands, centromeric regions, telomeric regions, and chromosome arms.

An “inter-repeat region” refers to the region of the chromosome or genomic DNA that is amplified by the Alu primers. It is also the region between the repetitive elements, and thus substantially free of Alu sequence.

Probes can also be specific to particular chromosome bands, where the probes are derived from processing microdissected bands or from cloned DNA localized to the band of interest. (See, e.g., Cheung et al., Nature, 409:953-958, 2001, for a discussion of the integration of cytogenetics landmarks into the sequence of the human genome.) The banding is a representation of the location of bands observed on Giemsa-stained chromosomes. The latter refers to the differential staining of chromosomes to elicit chromosome bands (the “G-bands”). Several different methods can be used to produce the G-banding pattern, e.g., pretreatment with a salt solution or with proteolytic enzymes (e.g., trypsin or pronase), followed by staining with Giemsa solution.

Chromosomes are typically displayed with the short arm first. Cytologically identified bands on the chromosome are numbered outward from the centromere on the short (p) and long (q) arms. At low resolution, bands are classified using the nomenclature [chromosome] [arm] [band], where band is a single digit. At a higher resolution, bands can be further subdivided into sub-bands, adding a second digit to the band number.

In additional to chromosomes and chromosomal segments, genomic DNA can also be utilized. This can include isolated or cloned DNAs, e.g., a specific region of DNA has been inserted into a vector or other nucleic acid carrier, such as a phage artificial chromosome (PAC), bacterial artificial chromosome (BAC), yeast artificial chromosome (YAC), or mammalian artificial chromosome (MAC). BAC clones can typically comprises inserts from about 100 kb to several hundred kb of genomic DNA; PAC of about 100 kb; and YAC of about 1,000 kb. These can be processed by the methods of the present invention to deplete the repetitive sequences. The depleted BAC clones (or PAC, YAC, or MAC) can be used for any purpose for which the clones are used. For instance, they can be used as FISH probes for the detection of chromosomal alterations in congenital diseases, prenatal diagnosis, tumor classification, diagnosis and prognosis evaluation, and chromosomal changes caused by radiation. They can also be arrayed on any suitable substrate, e.g., nitrocellulose, plastic, nylon, etc.

The probes can be produced by contacting chromosomal or genomic materials with a repeat primer (such as an end-Alu primer) under conditions effective for said primer to hybridize to a human Alu repetitive element. Contacting indicates that the probe and the chromosomal or genomic materials are physically combined in such a way that the probe, under suitable conditions, can anneal to the DNA. Such conditions include any conditions that are useful for hybridization (annealing) to occur, such as temperature (e.g., to dissociate the two strands of the chromosomal or genomic DNA), buffer, ionic strength, pH, etc.

The contacting can be achieved in any suitable environment. For example, the contacting can occur in a completely aqueous environment, where the primer is added to a solution comprising a microdissected chromosome segment. The segment or (genomic DNA) can also be affixed or associated with a substrate, such as an impermeable substrate (e.g., a glass slide or glass beads), or a permeable substrate (e.g., nitrocellulose or agar), when brought into contact with the probe. Examples of other substrates include, e.g., plastic (e.g., a multi-well plate), nylon and its derivatives.

An amplification step is performed after the probe is placed in contact with the chromosomal or genomic material. A preferred amplification is using polymerase chain reaction (“PCR”) where a DNA polymerase enzyme (such as Taq polymerase) is utilized to exponentially create copies of a template nucleic acid. PCR can be performed routinely, e.g., using conventional reaction conditions, substrates, and enzymes. The primers facilitate the amplification of DNA from the inter-repeat regions of said chromosome segment to produce the chromosome-specific DNA probe. The amplification is accomplished with a primer consisting of an end-Alu primer.

A chromosomal segment can be obtained by any suitable method. For example, microdissection can be utilized to physically excise regions of a chromosome for further processing to be used as probes. Microdissection can be accomplished routinely, e.g., using glass pipettes (using a micromanipulator), or laser microbeam. See, e.g., Guan et al., Genomics, 22:101-107, 1994; Meltzer et al., Nature Genet. 1:24-28, 1994; Lengauer et al., Cytogenetic Cell. Genet., 56: 27-30, 1991. The length of dissected DNA can vary, e.g., from about 5,000 to about 20,000 kb in size, depending on the size of the band. Amplification can also be performed in situ, prior to the microdissection step. See, e.g., U.S. Pat. No. 6,432,650.

Labeling of nucleic acids can be done routinely. For example, when PCR is utilized, modified substrate nucleotides can be utilized to facilitate the labeling procedures. Various modifications can be made to the polynucleotides, such as attaching detectable markers (avidin, biotin, radioactive elements, fluorescent tags and dyes, energy transfer labels, energy-emitting labels, binding partners, enzyme tags, colorimetric indicators, etc.). The polynucleotides can also be attached to supports, e.g., magnetic or paramagnetic microspheres (e.g., comprising ferromagnetic, supermagnetic, paramagnetic, superparamagnetic, iron oxide and polysaccharide), etc., which are then utilized as a support for a detectable label. The phrase “detectable label” refers to any material which facilitates the visualization of a probe when hybridized to a chromosome, including in the visible, or fluorescent wavelengths. See, e.g., U.S. Pat. No. 5,952,180 for use of energy transfer fluorescent labels.

Alu primers of the present invention can be 3′- and 5′-end Alu primers (or collectively, “end-Alu primers”). End-Alu primers are oligonucleotides primers which are substantially complementary to within about 50 base pairs (bp) of the 3′ or 5′ terminal nucleotide of the Alu repetitive element (e.g., within 50, 45, 40, 35, 30, 25 bp, etc.). A primer can be of any size and composition that is effective to initiate (“prime”) a polymerase reaction, and extend the primer using the chromosomal segment or genomic DNA as a template. Primers can be made of naturally-occurring nucleotides, derivatives thereof, and non-naturally-occurring nucleotides.

Alu repetitive elements are interspersed within the human genome with an average spacing of about every 4 kilo bases (kb) and an average size of about 300 bp. There are several hundred different Alu elements in the human genome. Alu sequences have as high as 900,000 copies within the human genome, and occupy about 5% of the total human genome. End-Alu primers can be designed to any of them, including to consensus sequences derived from aligning a plurality of family members. Examples of Alu sequences, include, e.g., Accession numbers U14573, U14571, U14572, U14568, etc. Other sequences can be identified routinely, e.g., by searching databases available at ncbi.nlm.nih.gov.

Examples of end-Alu primers that anneal to the 3′end of the Alu sequence, and which amplify DNA in a 5′ to 3′ direction, include:

| | | |
|---|---|---|
| AD-1: | 5′-ACA GAG YRA GAC TCY RTC TCA AC-3′ | (SEQ ID NO:1) |
| AD-2: | 5′-ACC AAC GAA TTC AGA CTC YRT CTC AAC-3 | (SEQ ID NO:2) |

Y = C or T; R = A or G

Examples of end-Alu primers that anneal to the 5′end of the Alu sequence, and which amplify DNA in a 5′ to 3′ direction, include:

| | | |
|---|---|---|
| AD3: | 5′-GTG AGC CAC CAC GCC CAG CC-3′ | (SEQ ID NO:3) |
| AD4: | 5′-ACC ACA GAA TTC CCA CCA CGC CCA GCC-3′ | (SEQ ID NO:4) |

Other useful primers include:

| | | |
|---|---|---|
| Alu-N1: | 5′-TTA CAG GYR TCA GCC ACY AC-3′; | (SEQ ID NO:5) |
| Alu-N2: | 5′-RCC AYT GCA CTY CAG CCT G-3′; | (SEQ ID NO:6) |

where Y = C or T; R = A or G.

Amplification conditions can be selected routinely, e.g., conventional conditions for carrying out PCR, such as denaturing DNA at about 90-95° C. for about 45-75 seconds; annealing with primer at 50-65° C. for about 30-90 seconds; and extending at 70-74° C. using a polymerase enzyme for about 30-90 seconds. Conditions can be such that the length of the amplification is about 3-6 kb, 3-5 kb, 3-4 kb, etc. A total of about 25-45 cycles can be used. When microdissected DNA is used, as little as one copy can be used, but preferably more than one, such at least five, 5-10, etc. The methods can be carried out without pretreatment, e.g., without an enzyme, such as topoisomerase 1 or pepsin.

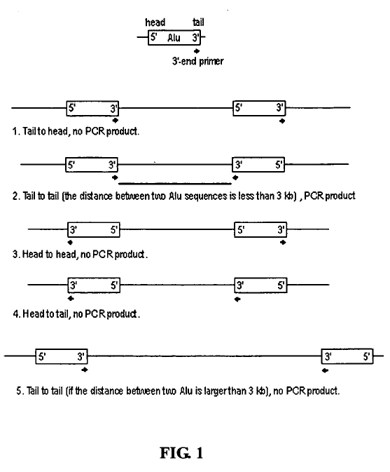

The amplifying can be accomplished with a primer consisting of the end-Alu primer. This indicates that a single primer is utilized in the amplification reaction. As shown in FIG. 1 for a 3′-end-Alu primer, only certain configurations at certain distances (limited by the efficacy of the PCR process) amplify DNA. Consequently, not all sequences are amplified (e.g., inter-Alu between head-to-head). However, that is not necessary since an objective is to produce a region-specific probe that is depleted of Alu sequences, not to amplify all sequences within the region of interest. To increase the number of amplified sequences, an amplification reaction can be additionally carried out on the same segment, but using a primer consisting of the 5′-end-Alu primer. Both products can be combined.

An additional advantage of using the end-Alu primers is that an Alu element within the two Alu sequences which serve as the template for the primer (see, FIG. 1) will block the PCR reaction.

Inter-Alu PCR can be also used to amplify unique sequences within a BAC clone. Using this method, unique sequences within a given BAC clone can be obtained for either Southern or FISH analysis. FISH probes produced in this way can be directly used to hybridize to both metaphase chromosomes and interphase nuclei.

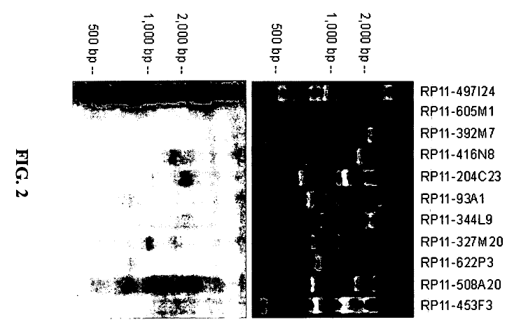

As shown in FIG. 2, AD2 can effectively amply BAC clone DNA which contains no repetitive sequences. DNA sequences from RP11-497124 could specifically hybridize the PCR products from RP11-497124, but did not hybridize to PCR products from other BAC clones although RP11-497124 contains a lot of repetitive sequences.

BAC (and other cloned genomic DNA) clones depleted of repetitive Alu elements can also be used to produce genomic arrays. The BAC clones produced by inter-Alu PCR of the present invention can be FISH-mapped (e.g., at a resolution of 1-2 MB) across the human genome, physically mapped to the genome using sequence tag sites (STS), and/or ordered using genetic linkage and/or radiation hybrid mapping data. These can then be utilized as FISH probes and/or to create genomic DNA microarrays. They can also be used, e.g., in comparative genomic hybridization. See, e.g., Ishkanian et al., Nature Genetics, 36, 299-303 (2004).

The genomic DNA can be arrayed using conventional gene chip technology. Basically, a DNA array or gene chip consists of a solid substrate upon which an array of DNA molecules have been attached. See, e.g., U.S. Pat. Nos. 5,744,305 or 6,054,270. The array can be ordered DNA array comprising a plurality of probes, where the probes are arranged in an identifiable or position-addressable pattern, e.g., such as the arrays disclosed in U.S. Pat. Nos. 6,156,501, 6,077,673, 6,054,270, 5,723,320, 5,700,637, WO09919711, WO00023803. The probes are associated with the solid support in any effective way. For instance, the probes can be bound to the solid support, either by polymerizing the probes on the substrate, or by attaching a probe to the substrate. Association can be, covalent, electrostatic, noncovalent, hydrophobic, hydrophilic, noncovalent, coordination, adsorbed, absorbed, polar, etc. When fibers or hollow filaments are utilized for the array, the probes can fill the hollow orifice, be absorbed into the solid filament, be attached to the surface of the orifice, etc. Solid substrates include, those with permeable or impermeable surfaces, such as glass, plastic, nylon, nitrocellulose, etc.

The probes can also be made single-stranded by using conventionally techniques, e.g., using RNA promoter primers (e.g., U.S. Pat. No. 5,545,522; Eberwine et al., 1992, Proc. Natl. Acad. Sci., 89, 3010-3014, 1992) or Qbeta Replicase (PCT/US87/00880).

Hybridization conditions for FISH analysis can be selected routinely, e.g., to ensure that the probe hybridizes specifically to the target chromosomal region. Generally, the probe is hybridized under effective conditions, e.g., the particular milieu in which the desired effect is achieved. Such a milieu includes, e.g., appropriate buffers, oxidizing agents, reducing agents, pH, co-factors, temperature, ion concentrations, formamide concentration, blocking DNA (e.g., sheared salmon sperm).

Analysis of hybridized probes can be done manually or using automated methods, e.g., a microscope is equipped with suitable software for analyzing the chromosome spreads. See, e.g., U.S. Pat. No. 6,221,607.

For other aspects of the polynucleotides, reference is made to standard textbooks of molecular biology. See, e.g., Hames et al., Polynucleotide Hybridization, IL Press, 1985; Davis et al., Basic Methods in Molecular Biology, Elsevir Sciences Publishing, Inc., New York, 1986; Sambrook et al., Molecular Cloning, CSH Press, 1989; Howe, Gene Cloning and Manipulation, Cambridge University Press, 1995; Ausubel et al., Current Protocols in Molecular Biology, John Wiley & Sons, Inc., 1994-1998.

Without further elaboration, it is believed that one skilled in the art can, using the preceding description, utilize the present invention to its fullest extent. The following preferred specific embodiments are, therefore, to be construed as merely illustrative, and not limiting of the disclosure in any way whatsoever. The entire disclosure of all applications, patents and publications, cited above are hereby incorporated by reference in their entirety.

### EXAMPLES

### Example 1

### Synthesis of Primer

The following primers are artificial synthesized:

| | | |
|---|---|---|
| AD-1: | 5′-ACA GAG YRA GAC TCY RTC TCA AC-3′; | (SEQ ID NO:1) |
| AD-2: | 5′-ACC AAC GAA TTC AGA CTC YRT CTC AAC-3′; | (SEQ ID NO:2) |

where Y = C or T; R = A or G.

### Example 2

### Chromosome Microdissection

Find the target chromosome under a microscope; dissect the target chromosomal region with glass needle controlled by a micromanipulator attached to the microscope. The dissected DNA is then transferred into collection solution containing AD-1 or AD-2 primer. Usually, 5-10 copies of target chromosomal region are used. After collecting enough copy numbers of dissected DNA fragments, the dissected DNA is amplified by PCR.

### Example 3

### PCR Amplification

About 5-10 copies of dissected DNA fragments is amplified in 50 μl PCR reaction solution (10 mM Tris-HCl, pH 8.4, 2 mM MgCl, 50 mM KCl, 0.1 mM gelatin, 200 mM DNTP, 0.5 mM AD-1 or AD-2 primer, and 2 Unit Taq polymerase).

The PCR reaction is as follows: 30-40 cycles at 92-95° C. for 45-75 seconds, 50-65° C. for 30-90 seconds, and 70-72° C. for 60-120 seconds. The PCR products can be checked by electrophoresis and their size distribution is ranged from 300 to 800 bp (FIG. 1).

### Example 4

### Fluorescence In situ Hybridization

FISH can be used to determine the accuracy of the chromosome microdissection. The process is as follows: Prepare FISH probe by adding 2 μl of first round PCR products into a 50 μl PCR reaction mixture identical to that described above except for the addition of 20 μM Biotin-16-dUTP for final concentration. The PCR reaction is continued for 20-30 cycles at 92-95° C. for 45-75 seconds, 50-65° C. for 30-90 seconds, and 70-72° C. for 60-120 seconds.

After the purification of probe by precipitation, the probe is resuspended in TE buffer. For each FISH reaction, 100-200 ng of probe is mixed with 10 μl hybridization mixture. After denaturation, the probe is hybridized to denatured metaphase chromosomes, and visualized.

### Example 5

### Amplification of BAC DNA with Alu Primers

(1) Culture BAC clone: plating BAC onto a plate, culture at 37° C. for 12 hours, select a single colony of the BAC and culture it into a culture medium by shaking. After culture, the BAC DNA is prepared according to standard methods.

(2) The method to amplify BAC DNA without repetitive sequences is as follows: The PCR reaction is carried out in 50 μl PCR reaction buffer (10 mM Tris-HCl, pH8.4, 2 mM MgCl, 50 mM KCl, 0.1 mM gelatin, 200 mM dNTP, 0.5 mM Alu-N1 or Alu-N2 primers (or other suitable end-Alu primers), 2 units Taq polymerase) with 50 ng BAC DNA as template. PCR reaction is as follows: 30 cycles at 94° C. for 1 min, 60° C. for 1 min, and 72° C. for 2 min.

### Example 6

### Southern Blot Analysis of PCR Products

After electrophoresis, the PCR products in Example 5 were transferred on to a nylon membrane. DNA from a BAC clone (RP11-110O7) was labeled with ³²P and hybridized to the DNA on the membrane by Southern blot hybridization. Southern blot hybridization result is shown in FIG. 2. The probe used in this study was a whole BAC clone which contains repetitive sequences.

### Example 7

### Storage, Screening and Identification of PCR Products

The above-mentioned PCR products can be cloned into TA-vector to generate a genomic DNA library in order to store and amplify these PCR products. Alu-negative colonies can be screened using BAC clone DNA (RP11-11007, sized 120 kb). The inserts of selected colonies can be amplified by PCR using vector flank specific primers. Some of the cloned PCR products were sequencing analyzed. Sequencing results showed that these PCR products were ranged from 400 to 2,000 bp in size (average 800 bp) and contains no repetitive sequences. (An RNA polymerase promoter-primer can be included in the vector to produce single-stranded RNA, if desired.)

### Example 8

### Preparation of FISH Probe

The PCR products can be used as FISH probe by labeling with fluorescent dye. The probe is labeled by random priming method which can increase the labeled DNA.
(1) Suspend 100 ng DNA in 24 μl water and put it on ice, add 20 μl random priming buffer (2.5×). Mix the solution and boil it for 5 min, and transfer it on the ice.
(2) Add 5 μl 10× dNTP and mix the solution, add 1 μl enzyme solution.
(3) After mixture, the reaction is incubated at 37° for 1 h. Check the labeling products by running 2 μl of product. The labeling is considered good if the sizes of probes are ranged from 200 to 2,000 bp.
(4) Inactivation of the labeling reaction by heating at 750 for 10 min, or adding 5 μl stop buffer.

The entire disclosures of all applications, patents and publications, cited herein and of corresponding U.S. application Ser. No. 10/489,759, filed Mar. 17, 2004; PCT/CN01/01209 filed on Jul. 27, 2001 and PCT/CN2002/000580 filed on Aug. 22, 2002 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method of preparing a chromosome-specific DNA probe, comprising:
contacting a chromosome segment or genomic DNA with an end-Alu primer under conditions effective for said primer to hybridize to a human Alu repetitive element, and
amplifying the DNA from the inter-repeat regions of said chromosome segment or genomic DNA to produce the chromosome-specific DNA probe, wherein said amplifying is accomplished with a primer consisting of said end-Alu primer.

2. A method of claim 1, wherein chromosome segment is a chromosome arm.

3. A method of claim 1, wherein said chromosome segment is a chromosome G-band.

4. A method of claim 1, wherein said chromosome segment is a telomere.

5. A method of claim 1, wherein said chromosome segment is obtained by micro dissection.

7. A method of claim 1, wherein said genomic DNA is a BAC clone.

8. A method of claim 1, wherein the end-Alu primer is a 3′-end-Alu primer which comprises:

| | | |
|---|---|---|
| AD-1: | 5′-ACA GAG YRA GAC TCY RTC TCA AC-3′, | (SEQ ID NO:1) |
| or | | |
| AD-2: | 5′-ACC AAC GAA TTC AGA CTC YRT CTC AAC-3. | (SEQ ID NO:2) |

9. A method of claim 1, wherein the end-Alu primer is a 5′-end-Alu primer which comprises:

| | | |
|---|---|---|
| AD3: | 5′-GTG AGC CAC CAC GCC CAG CC-3′, | (SEQ ID NO:3) |
| or | | |
| AD4: | 5′-ACC ACA GAA TTC CCA CCA CGC CCA GCC-3′. | (SEQ ID NO:4) |

10. A method of claim 8, wherein the genomic DNA is a BAC clone.

11. A method of claim 9, wherein the genomic DNA is a BAC clone.

12. A method of claim 8, wherein said chromosome segment is obtained by microdissection.

13. A chromosome-specific DNA probe produced by a method of claim 1.

14. A DNA microarray, comprising a plurality of ordered DNAs produced by a method of claim 1.
